(19) United States
(12) Patent Application Publication
(10) Pub. No.: US 2024/0277736 A1
(43) Pub. Date: Aug. 22, 2024

(54) USE OF PSILOCYBIN IN THE TREATMENT OF NEUROLOGICAL BRAIN INJURY AND MIGRAINES

(71) Applicant: Revive Therapeutics Ltd, Toronto (CA)

(72) Inventor: Fabio Andrea Chianelli, Toronto (CA)

(73) Assignee: Revive Therapeutics Ltd, Toronto, ON (CA)

(21) Appl. No.: 17/919,420

(22) PCT Filed: Mar. 18, 2021
(86) PCT No.: PCT/CA2021/050360

§ 371 Date: Oct. 17, 2022

Related U.S. Application Data
(60) Provisional application No. 63/011,493, filed on Apr. 17, 2020.

(51) Int. Cl. A61K 31/675; A61P 25/00

(52) U.S. Cl. CPC A61K 31/675; A61P 25/00

(57) ABSTRACT

Methods for the treatment of a brain injury or a migraine in a mammal including administering a therapeutically effective amount of psilocybin or a pharmaceutically acceptable salt or solvate thereof, to a mammal in need thereof. Use of a pharmaceutical composition including psilocybin or a pharmaceutically acceptable salt or solvate thereof, together with one or more pharmaceutically acceptable carriers, diluents and excipients for the treatment of a brain injury or a migraine in a mammal.

### FIELD

The present invention relates to pharmaceutical compositions comprising psilocybin and their use for the treatment of neurological brain injuries and migraines.

### BACKGROUND

Psilocybin (4-phosphoryloxy-N,N-dimethyltryptamine) is a substituted indolealkylamine and belongs to the group of hallucinogenic tryptamines. Psilocybin is a prodrug and undergoes dephosphorlyation to Psilocin in vivo. The chemical formulas for Psilocybin and Psilocin are:

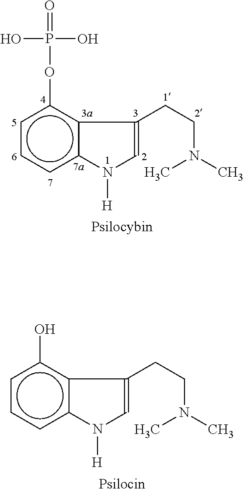

Psilocybin was distributed worldwide under the name Indocybin® (Sandoz) as a short-acting and more compatible substance (than, for example, LSD) to support is use as a psychotherapeutic. Experimental and therapeutic use was extensive and without complications. (1)

Brain injury from a concussion is a complex condition which causes structural damage and functional deficits from primary and secondary injury mechanisms, respectively. (2) The primary injury mechanism is the result of the immediate mechanical disruption of brain tissue that occurs at the time of exposure to the external force and includes, damage to blood vessels (hemorrhage), and axonal shearing, in which the axons of neurons are stretched and torn. The secondary injury mechanism evolves over minutes to months after the primary injury, and is the result of cascades of metabolic, cellular and molecular events that ultimately lead to brain cell death, tissue damage and atrophy in the injury boundary zone and subcortical regions.

Even in situations where there is minimal brain injury from a concussion, cognitive deficits, e.g., loss of memory, movement, sensation (e.g., vision or hearing) or emotional functioning (e.g., personality changes, depression) can result. Delayed symptoms such as irritability and other personality changes, sensitivity to light and noise, sleep disturbances, psychological adjustment problems such as depression and disorders of taste and smell may also result. (2)

Typically for mild forms of brain injury, e.g. concussion, bed rest, fluids, and a mild pain reliever such as acetaminophen (Tylenol) may be prescribed. Ice may be applied to bumps to relieve pain and decrease swelling. Cuts are numbed with medication such as lidocaine, by injection or topical application. If needed, the wound usually is closed with skin staples, stitches (sutures), or, occasionally, a skin glue called cyanoacrylate (Dermabond).

However, there is no available treatment for the neurological brain damage and the associated cognitive deficits experienced. (2)

Migraine is a common disabling primary headache disorder. Epidemiological studies have documented its high prevalence and high socio-economic and personal impacts all over the world (Fendrich et al., Cephalalgia, 2007; 27:347-54; Le et al., BMJ Open, 2012; 2(4); Yong et al., J Headache Pain. 2012; 13:303-10; Yoon et al., J Headache Pain. 2012; 13:215-23; Ertas et al., J Headache Pain. 2012; 13:147-57). Migraine is now ranked by the World Health Organization as number 19 among all diseases world-wide causing disability.

Commonly starting at puberty, migraine most affects those aged between 20 and 50 years but can trouble much younger people, including children. The one-year prevalence in adults is estimated to be 15%. In children and adolescents the prevalence is approximately 5%. European and American studies have shown that 6-8% of men and 15-18% of women experience migraine each year. The higher rates in women everywhere (2-3 times those in men) are hormonally-driven. Prevalence declines after 50 years of age (WHO Fact Sheet N° 277, 2004; EMA CHMP Guideline, 2007).

There is no absolute cure for migraine since its pathophysiology has yet to be fully understood (Pietrobon & Striessnig, Nat Rev Neurosci. 2003; 4:386-98; Cucchiara & Detre, Med Hypotheses. 2008; 70:860-5). There are two medication strategies for treating migraine headaches. Treating the pain at the onset offers the best relief. Over-the-counter pain relievers such as acetaminophen, aspirin or other nonsteroidal anti-inflammatory drugs (NSAIDs) such as ibuprofen are commonly used (Pardutz & Schoenen, Pharmaceuticals. 2010; 3:1966-1987). Prescription drugs such as triptans are used for headaches not relieved by over-the-counter medications. These are generally not applied to people who have high blood pressure or a heart disease. For those whose headaches are not adequately relieved with these medications, the second medication strategy involves medications prescribed prophylactically. These are normally prescribed to treat other disorders but have been successful at reducing the frequency or severity of migraine headaches. Blood pressure medications such as beta-blockers or calcium channel blockers; antidepressant medications such as amitriptyline or venlafaxine; and anticonvulsant medications such as divalproex or topiramate (Hildreth et al., JAMA. 2009; 301:2608) have been used.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

### SUMMARY OF THE INVENTION

The present disclosure, in one aspect, relates to a method for the treatment of a brain injury or a migraine in a mammal comprising administering a therapeutically effective amount of psilocybin or a pharmaceutically acceptable salt or solvate thereof, to a mammal in need thereof.

The present disclosure, in another aspect, relates to a use of a pharmaceutical composition including psilocybin or a pharmaceutically acceptable salt or solvate thereof, together with one or more pharmaceutically acceptable carriers, diluents and excipients for the treatment of a brain injury or a migraine.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic diagram of a timeline of an in vitro experiment, where +AO is with antioxidants and −A. O. is without antioxidants;

FIG. 2 is a schematic diagram of a timeline of an in vivo experiment;

FIG. 3A are photomicrographs representing MAP2 immunostaining;

FIG. 3B is a bar graph of MAP2 immunoreactivity;

FIG. 4A is a bar graph showing TBI significantly reduced duration stayed in the target zone;

FIG. 4B is a bar graph showing the average number of platform crossings;

FIG. 4C is a bar graph showing movement speed on PID3; and

FIG. 5A, FIG. 5B and FIG. 5C are graphs showing high dose Psilocybin (PSI) treatment significantly increased the time in the target area and the average number of platform crossings in TBI mice.

### DETAILED DESCRIPTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

For use in therapy a therapeutically effective amount of the psilocybin or pharmaceutically acceptable salts or solvates thereof, may be presented as a pharmaceutical composition. Thus, in a further embodiment the invention provides a pharmaceutical composition of psilocybin or pharmaceutically acceptable salts or solvates thereof in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

When applicable, the compositions of the present invention, including psilocybin may be in the form of and/or may be administered as a pharmaceutically acceptable salt.

Typically, a pharmaceutically acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, nitrate, phosphate, hydrogen phosphate, dihydrogen phosphate acetate, maleate, malate, fumarate, lactate, tartrate, citrate, formate, gluconate, succinate, pyruvate, oxalate, oxaloacetate, trifluoroacetate, saccharinate, benzoate, methanesulphonate, ethanesulphonate, benzenesulphonate, p-toluenesulphonate and isethionate.

Suitable salts may also be formed from bases, forming salts including ammonium salts, alkali metal salts such as those of sodium and potassium, alkaline earth metal salts such as those of calcium and magnesium.

Pharmaceutically acceptable salts may also be prepared from other salts, including other pharmaceutically acceptable salts, using conventional methods.

Those skilled in the art of organic or coordination chemistry will appreciate that many organic and coordination compounds can form complexes with solvents in which they are reacted or from which they are precipitated or crystallized. These complexes are known as “solvates”. For example, a complex with water is known as a “hydrate”. Solvates of psilocybin are within the scope of the present invention.

Pharmaceutical compositions of the invention may be formulated for administration by any appropriate route, for example by the oral (including buccal or sublingual). Therefore, the pharmaceutical compositions of the invention may be formulated, for example, as tablets, capsules, powders, granules, lozenges, creams or liquid preparations, such as oral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan, monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

It should be understood that in addition to the ingredients particularly mentioned above, the formulations may include other agents conventional in the art having regard to the type of formulation in question.

The compositions of the present invention may be suitable for the treatment of diseases in a human or animal patient. In one embodiment, the patient is a mammal including a human, horse, dog, cat, sheep, cow, or primate. In one embodiment the patient is a human. In a further embodiment, the patient is not a human.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In certain embodiments of the present invention, pharmaceutically acceptable compositions of the present disclosure can be administered to humans and other animals at doses within the range of about 0.5 mL/day to about 3.0 mL/day and at a concentration within the range of about 0.5 mM to about 3.0 mM, particularly within the range of about 0.5 mL/day to about 3.0 mL/day and at a concentration within the range of about 0.5 mM to about 1.0 mM, particularly within the range of about 0.5 mL/day to about 3.0 mL/day and at a concentration within the range of about 0.5 mM to about 1.5 mM, particularly within the range of about 0.5 mL/day to about 3.0 mL/day and at a concentration within the range of about 0.5 mM to about 2.0 mM, particularly within the range of about 0.5 mL/day to about 3.0 mL/day and at a concentration within the range of about 0.5 mM to about 2.5 mM, particularly within the range of about 0.5 mL/day to about 1.0 mL/day and at a concentration within the range of about 0.5 mM to about 1.0 mM, particularly within the range of about 0.5 mL/day to about 1.5 mL/day at a concentration within the range of about 0.5 mM to about 1.0 mM, particularly within the range of about 0.5 mL/day to about 2.0 mL/day at a concentration within the range of about 0.5 mM to about 1.0 mM, particularly within the range of about 0.5 mL/day to about 2.5 mL/day at a concentration within the range of about 0.5 mM to about 1.0 mM, particularly within the range of about 0.5 mL/day to about 3.0 mL/day at a concentration within the range of about 0.5 mM to about 1.0 mM, particularly within the range of about 0.5 mL/day to about 1.0 mL/day at a concentration within the range of about 0.5 mM to about 1.5 mM, particularly within the range of about 0.5 mL/day to about 1.0 mL/day at a concentration within the range of about 0.5 mM to about 1.5 mM, particularly within the range of about 0.5 mL/day to about 2.0 mL/day and at a concentration within the range of about 0.5 mM to about 1.5 mM, particularly within the range of about 0.5 mL/day to about 2.5 mL/day at a concentration within the range of about 0.5 mM to about 1.5 mM, particularly within the range of about 0.5 mL/day to about 3.0 mL/day at a concentration within the range of about 0.5 mM to about 1.5 mM, particularly within the range of about 0.5 mL/day to about 1.0 mL/day at a concentration within the range of about 0.5 mM to about 2.0 mM, particularly within the range of about 0.5 mL/day to about 1.5 mL/day and at a concentration within the range of about 0.5 mM to about 2.0 mM, particularly within the range of about 0.5 mL/day to about 2.0 mL/day at a concentration within the range of about 0.5 mM to about 2.0 mM, particularly within the range of about 0.5 mL/day to about 2.5 mL/day at a concentration within the range of about 0.5 mM to about 2.0 mM, particularly within the range of about 0.5 mL/day to about 3.0 mL/day and at a concentration within the range of about 0.5 mM to about 2.0 mM, particularly within the range of about 0.5 mL/day to about 1.0 mL/day at a concentration within the range of about 0.5 mM to about 2.0 mM, particularly within the range of about 0.5 mL/day to about 1.5 mL/day and at a concentration within the range of about 0.5 mM to about 2.0 mM, particularly within the range of about 0.5 mL/day to about 2.0 mL/day and at a concentration within the range of about 0.5 mM to about 2.0 mM, particularly within the range of about 0.5 mL/day to about 2.5 mL/day at a concentration within the range of about 0.5 mM to about 2.0 mM, particularly within the range of about 0.5 mL/day to about 3.0 mL/day and at a concentration within the range of about 0.5 mM to about 2.0 mM, particularly within the range of about 0.5 mL/day to about 1.0 mL/day and at a concentration within the range of about 0.5 mM to about 2.5 mM, particularly within the range of about 0.5 mL/day to about 1.5 mL/day at a concentration within the range of about 0.5 mM to about 2.5 mM, particularly within the range of about 0.5 mL/day to about 2.0 mL/day and at a concentration within the range of about 0.5 mM to about 2.5 mM, particularly within the range of about 0.5 mL/day to about 2.5 mL/day and at a concentration within the range of about 0.5 mM to about 2.5 mM, particularly within the range of about 0.5 mL/day to about 3.0 mL/day at a concentration within the range of about 0.5 mM to about 2.5 mM, particularly a dose of 0.5 mL/day at a 2 mM concentration, particularly a dose of 1.0 mL/day at a 2 mM concentration, particularly a dose of 1.2 mL/day at a 2 mM concentration, particularly a dose of 1.5 mL/day at a 2 mM concentration, particularly a dose of 2.0 mL/day at a 2 mM concentration, particularly a dose of 2.5 mL/day at a 2 mM concentration, particularly a dose of 3 mL/day at a 2 mM concentration, particularly a dose within the range of about 1.8 mL/day to about 2.2 mL/day at a concentration within the range of about 1.8 mM to about 2.2 mM, particularly a dose within the range of about 1.9 mL/day to about 2.1 mL/day at a concentration within the range of about 1.9 mM to about 2.1 mM, particularly within the range of about 0.5 mL/day to about 3.0 mL/day at a 2 mM concentration, particularly up to 3 mL/day at a concentration up to 3.0 mM, particularly up to 3 mL/day at a 2 mM concentration, and this should provide a therapeutically effective dose. However, the daily dose will necessarily be varied depending upon the host treated, the particular route of administration, and the severity of the illness being treated. Accordingly, the optimum dosage may be determined by the practitioner who is treating any particular patient. In another embodiment, the optimal dose may be higher.

As used herein the term “treatment” refers to defending against or inhibiting a symptom, treating a symptom, delaying the appearance of a symptom, reducing the severity of the development of a symptom, and/or reducing the number or type of symptoms suffered by an individual, as compared to not administering a pharmaceutical composition of the invention. The term treatment encompasses the use in a palliative setting

Psilocybin is a strong agonist of the 5-HT2A receptor, as well as, a moderate agonist at 5-HT1A and 5-HT2C.3 receptors. The 5-HT2A receptors are located within the thalamus and cortex of the brain. Activation of 5-HT2A receptors in the thalamus, the area of the brain responsible for sensory input, appears to decrease thalamic activity, thus leading to sensory alterations commonly referred to as hallucinations. (3) Due to this alteration in sensory perception and serotonergic activity of psilocybin, much of the research for this agent has been focused on those mental health conditions with abnormalities in sensory perception, such as depressive disorders and anxiety or anxiety-related disorders. Note that these symptoms sometimes occur in concussions. Psilocybin has also been researched for use in substance use disorders. (3)

However, 5-HT2A activity does not appear to account fully for psilocybin's effect. There is growing evidence that psilocybin might also be beneficial in treating limiting brain injury through its potential to contribute to brain complexity and plasticity. (4) Therefore, the present inventor postulates that psilocybin can reduce or eliminate the common cognitive and sensory deficit symptoms resulting from concussion through its 5-HT2A activity, as well as, help repair the limited brain injury resulting from a concussion by its contributing to brain complexity and plasticity, as well as, its capability to stimulate neurogenesis.

Scientists at the at University of California, Davis (UC Davis) have reported that psychedelic drugs (including psilocybin) promote neural plasticity and development (4). The UC Davis scientists treated cultures of cortical neurons with psychedelics and observed that the neurons developed and increased in complexity. They also saw these results in the brains of fly larvae and zebrafish, indicating that psychedelics also have a tangible effect in living organisms. In a separate experiment by the UC Davis scientists, psychedelics were found to significantly increase the number of dendritic spines on cortical neurons. Dendritic spines form synapses with other neurons and are a major site of molecular activity in the brain. Electrophysiological recordings found that the frequency and strength of neural currents were increased for many hours after the psychedelic compounds had been removed. Therefore, psychedelics may have the potential to produce both structural and functional effects on neurons. (4)

The UC Davis scientists also attempted to determine the mechanism by which the structural and functional effects occurred. It has been established that mTOR regulates neuronal development and plasticity and that its activity is disturbed in neurodevelopmental and neurodegenerative diseases. (6) mTOR therefore was blocked and it was observed that the psychoplastogenic effects discussed above were inhibited, indicating that psychedelics may activate mTOR making this a potential mechanism for the neurogenesis activity. (4)

The UC Davis study builds on previous findings by the Beckley/Sant Pau Research Programme, which observed that components of the psychedelic brew ayahuasca promoted growth and maturation of neurons. (7) The study also builds upon reports in the literature from the 1950s, where it was found that LSD reversed the sedating effects of phenobarbital in cats (5, 8).

Based at least on the above results, the present inventor has a sound basis for predicting that neuron damage caused by a concussion can be mitigated through psilocybin's promotion of structural and functional neural plasticity and development for treating traumatic brain injury (mild-TBI=concussion), more severe types of TBI, stroke and Alzheimer's.

Migraines are debilitating headaches caused by neurologic stimulation of blood vessel dilation in the brain.₉ While they can be triggered by stress, anxiety, fatigue or depression, the root biological cause is unclear. Migraines carry a significant burden and socioeconomic impact, having been found in 2013 to be the 6^{th} leading cause of years lost to disability.₁₀ Current therapies, including over-the-counter pain relievers are generally unsatisfactory in the relief of symptoms, and poor understanding of the biological cause has hampered the discovery of effective therapies for migraines.₉ Treatment of chronic or episodic migraines may also be approached with preventive drugs. Hemiplegic migraines, a type that is associated with weakness on one side of the body, are especially difficult to treat because of concerns about vessel spasm and stroke.₁₁ A lack of good treatments for acute hemiplegic migraine makes prevention using safe daily administration of prophylactic compounds especially important.

Psilocybin is a strong activator of serotonin receptors, particularly 5-HT2, ₁₂ which is a main mediator of serotonin signaling in the part of the brain known as the hypothalamus.₁₃ Irregularities in the neurotransmitter serotonin have long been known to be associated with chronic headaches involving brain vasculature, including migraines.₁₄ In fact, serotonin deficiency has been discovered in families with genetic predisposition to hemiplegic migraines.₁₅ Such signaling in the thalamus and hypothalamus regulate sensory input and related disorders including anxiety and depression, which psilocybin has successfully been used to treat.₁₆ Low-dose psilocybin and other serotonin receptor activators can prevent another debilitating chronic headache disorder known as cluster headache (CH) and even induce remission.₁₇ CH is characterized by abnormal connectivity of the hypothalamus as has been demonstrated by fMRI brain imaging.₁₈ Furthermore, direct stimulation of the hypothalamus effectively eliminated CH in 10 of 16 patients and significantly reduced them in the other 6 in one study.₁₉ Similarly, the hypothalamus has been shown to be a mediator of chronic migraines as also evidenced by fMRI data.₂₀ Thus, parallel brain activity irregularities involving the hypothalamus are likely to be at play in both CH and chronic migraines and are modulated by serotonin agonists, including psilocybin.

Psilocybin has proven safety in clinical doses that effectively mediate its neurological effects. Psilocybin has been extensively studied in clinical trials and distributed worldwide as a clinical therapy for anxiety and depression with a very safe toxicity profile, even with unsupervised administration.₉ Based at least in part on the foregoing, and together with a mechanism of action on hypothalamic serotonin receptors, as observed in cluster headache, the present inventor has a sound basis for predicting that psilocybin is an effective drug for preventing or treating migraines, including those that are difficult to treat and require preventive therapies.

### 1. Summary of Experimental Study:

A study was designed to evaluate the therapeutic effect of Psilocybin in a rodent model of traumatic brain injury (TBI) and neuronal culture.

Psilocybin is a 5HT2a psychedelics, which increase BDNF expression and neuritogenesis. These responses may improve neural repair after traumatic brain injury. In the current study, we conducted in vitro and in vivo experiments to characterize the therapeutic effect of Psilocybin in TBI animals.
(i) Neuroprotective effect of Psilocybin in primary cortical neuronal culture.
   Cultured cells were treated with glutamate to simulate glutamate overflow in brain injury. Glutamate significantly reduced MAP-2 (microtubule-associated protein 2, a neuronal marker) immunoreactivity. This reaction was not significantly altered by Psilocybin.
(ii) Neuroreparative effect of Psilocybin in a mouse model of traumatic brain injury.
   Adult mice were randomly assigned to 4 groups: (1) control, (2) TBI+veh, (3) TBI+low dose Psilocybin, and (4) high dose Psilocybin. TBI was delivered by a Controlled cortical impact (CCI). Vehicle (saline) or Psilocybin was given intranasally injection (i.n.) starting from 4 days after TBI for 5 days. Cognitive function was examined by the Morris water maze test after the injury. Animals were sacrificed for PCR or protein analysis after the behavior test. We found that Psilocybin at a high dose significantly improved cognitive function in the TBI mice.
   These data support that Psilocybin, given after the injury, improves cognitive function in TBI mice.
2. Objective: This study was designed to evaluate the therapeutic effect of Psilocybin in a rodent model of traumatic brain injury and neuronal culture.

### 3. Experimental Study Details:

### 3-1: Methods and Material:

### A. Primary Cultures of Rat Cortical Neurons (PCN)

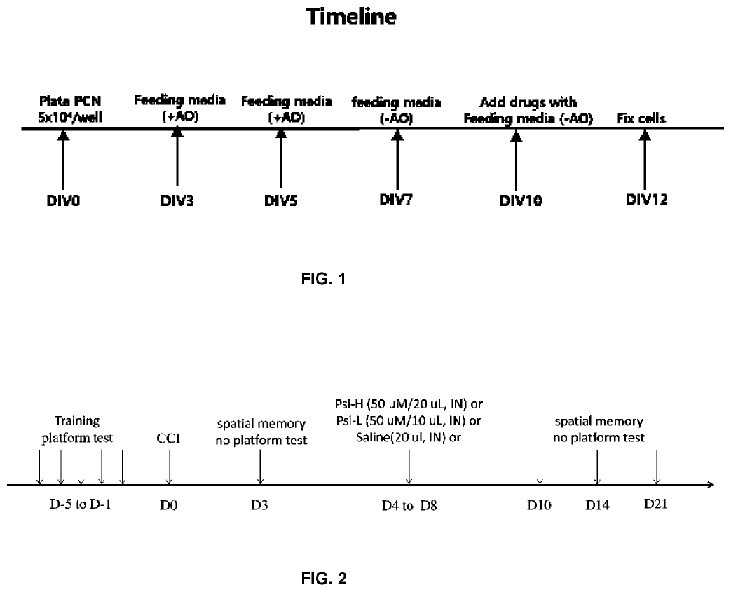

Primary cultures were prepared from embryonic (E14-15) cortex tissues obtained from fetuses of timed pregnant Sprague-Dawley rats as we previously described (1). The olfactory bulbs, striatum, and hippocampus was removed aseptically; cortices were dissected. After removing the blood vessels and meninges, pooled cortices were trypsinized (0.05%; Invitrogen, Carlsbad, CA) for 20 min at room temperature. After rinsing off trypsin with pre-warmed Dulbecco's modified Eagle's medium (Invitrogen), cells were dissociated by trituration, counted, and plated into 96-well (5.0×10⁴/well) cell culture plates precoated with poly-d-lysine (Sigma-Aldrich). The culture plating medium consisted of neurobasal medium supplemented with 2% heat-inactivated fetal bovine serum (FBS), 0.5 mM L-glutamine, 0.025 mM L-glutamate and 2% B27 (Invitrogen). Cultures were maintained at 37° C. in a humidified atmosphere of 5% CO₂ and 95% air. The cultures were fed by exchanging 50% of media with feed media (Neurobasal medium, Invitrogen) with 0.5 mM L-glutamate and 2% B27 with antioxidants supplement on days in vitro (DIV) 3 and 5. On DIV 7 and 10, cultures were fed with media containing B27 supplement without antioxidants (Invitrogen). On DIV 10, cultures were treated with reagents. After 48 hrs, cells were fixed at 4% paraformaldehyde for 1 hour at room temperature (please see the timeline in FIG. 1).

### B. Immunocytochemistry

Cells were fixed 48 hours after treatment of reagents using 4% P.F.A. After removing 4% P.F.A. solution, cells were washed with phosphate-buffered saline (PBS). Fixed cells were treated with blocking solution [5% bovine serum albumin (B.S.A.) and 0.1% Triton X-100 (Sigma, St. Louis, MO, U.S.A.) in PBS] for 1 hour. The cells were incubated for 1 day at 4° C. with a mouse monoclonal antibody against MAP2 (1:500, Millipore, Billerica, MA, U.S.A.) and then rinsed three times with PBS. The bound primary antibody was visualized using Alexa Fluor 488 goat anti-mouse secondary (Invitrogen). Images were acquired using a camera DS-Qi2 (Nikon, Melville, NY) attached to a NIKON ECLIPSE Ti2 (Nikon, Melville, NY). Data were analyzed using N.I.S. Elements AR 5.11 Software (Nikon).

### C. Animals:

Adult male CD1 mice were used for this study. Animals were randomly assigned to 4 groups: (1) naïve (n=9), (2) TBI+veh (n=9), (3) TBI+low dose Psilocybin (n=7), and (4) high dose Psilocybin (n=7).

### D. Controlled Cortical Impact (CCI) Surgery and Drug Treatment

CCI: Mice were anesthetized with isoflurane and placed in a stereotaxic frame. A midline incision was made to expose the skull, and a 4 mm craniotomy was made centered at −2 mm posterior to bregma and 0.5 mm lateral to midline over the left hemisphere. Mice were subjected to CCI at a 1.0 mm impact depth and a nominal velocity of 5 m/s. The dwell time was 500 ms, and the tip size was 2 mm. A computer-controlled pneumatically-driven piston from the CCI impactor device (TBI-0310 Impactor, Precision Systems and Instrumentation, Fairfax Station, VA) was used to impact the brain. After the impact, the head wound was sutured. Body temperature was maintained at 37° C. using a temperature-controlled incubator. Control animals received sham surgery, including craniotomy without cortical impact.

Intranasal drug delivery: Animals were anesthetized with isoflurane each day and were placed in a supine position. Psilocybin (high dose: 50 UM in 20 μl saline; low dose: 50 UM in 10 μl saline, Cayman Chemical, Michigan, U.S.A.) or saline (20 μl) was delivered into nostrils of each mouse per day from day 4 to day 8 (total 5 days) after CCI. No animal died during surgery or during post-TBI drug treatment.

### E. Morris Water Maze Test:

Cognitive function was assessed using the Morris water maze (MWM) test. A circular tank 200 cm in diameter, 45 cm in height, was filled with water maintained between 25-28° C. All animals received 5-day training prior to the T.B.I. (see timeline, FIG. 2). In the training period, the water level was lowered in the tank so that the surface of the platform (8 cm in diameter) was 1.5 cm above the water level. The animal was allowed to swim for 60 seconds in the pool to locate the platform. After the platform was located, the animal was allowed to remain on the platform for 30 seconds. If the animal did not locate the platform within 60 seconds, it was gently guided to it by the experimenter and allowed to remain for 30 seconds.

On post-injury days (PID) 3, 10, 14, and 21, animals were evaluated in 60 s probe trials without the escape platform. The swim path of a mouse during each trial will be recorded by a video camera connected to a tracking system. Latency time and the length of swim path were recorded. The locomotor activity of the mice was analyzed using an average swim speed. The spatial memory for the platform location during probe trials was evaluated by the analysis of the dwelling duration (in sec) and the number of times the animal crossed the platform zone, defined as 3× the diameter of the platform (i.e., 24 cm diameter, or an additional 8 cm radius beyond the platform perimeter). All parameters were automatically recorded and analyzed by video tracking software (Etho vision XT 8.5, Noldus, Leesburg, VA, U.S.A.).

### G. Statistical Analysis

Data were presented as mean+s.e.m. One or two-way ANOVA and post-hoc Fisher tests were used for statistical comparisons, with a significance level of p<0.05.

### 3-2: Results

### A. Psilocybin (PSI) Did not Significantly Protect Against Glutamate Neurotoxicity in Primary Cortical Neuronal Culture

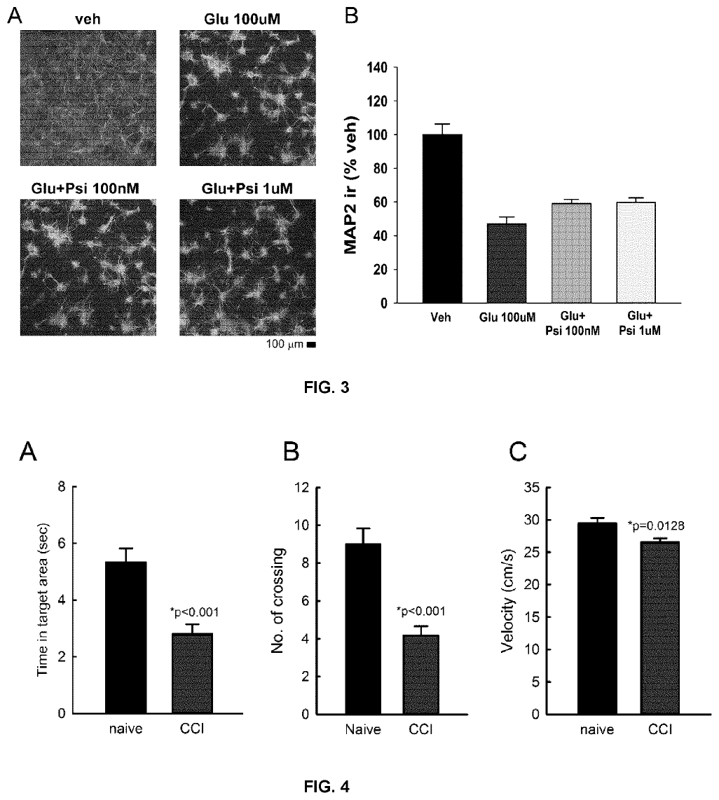

Glutamate (Glu)-mediated neuronal loss was examined by MAP-2 immunostaining. Typical photomicrographs were shown in FIG. 3A. The MAP2 immunoreactivity (MAP2-ir) was quantified and averaged to the mean of vehicle control group (FIG. 3B). Glu (100 UM) significantly reduced MAP2-ir (FIG. 3B1, Glu vs. veh, p<0.001, F_{3,19}=29.361, one-way ANOVA+ post hoc Fisher test). There is a non-significant trend that Psilocybin may partially antagonize GLU-mediated neurodegenerative response (p=0.062, Glu vs. Glu+100 nM PSI.; p=0.061, Glu vs. Glu+1 μM PSI; FIG. 3B).

With reference to FIG. 3, Psilocybin (Psi) did not antagonize glutamate (Glu)-mediated neuronal loss in primary cortical neuronal culture (PCN). FIG. 3 (A) Representing MAP2 immunostaining. FIG. 3(B) The MAP2-ir was averaged to the mean of vehicle control group. Glu treatment significantly reduced MAP2-ir (p<0.001). Glu-mediated MAP2-ir reduction was not significantly antagonized by Psilocybin (p>0.05, one-way ANOVA+post-hoc Fisher test). Data are represented as mean+/−S.E.M., n=5-6 per each group.

### B. Water Maze Test:

A total of 32 mice were used for this study. Adult mice were randomly assigned to 4 groups: (1) naïve (n=9), (2) TBI+veh (n=9), (3) TBI+low dose Psilocybin (n=7), and (4) high dose Psilocybin (n=7).

On post-injury day 3 (PID3) before drug treatment, animals received a single 60-second water maze test with the platform removed. As seen in FIG. 4, TBI significantly reduced (A) duration stayed in the target zone, (b) the average number of platform crossings, and (c) movement speed. These data can also be found in Table 1-3.

TABLE 1
Time in the target area in MWM test on PID3 (before drug
treatment) and PIDs 10, 14, 21 (after drug treatment)

| | Time in the target area (sec) | | | | |
|---|---|---|---|---|---|
| | n= | Day 3 | Day 10 | Day 14 | Day 21 |
| Naïve | 9 | 5.33 ± 0.49 | 5.97 ± 0.54 | 3.75 ± 0.55 | 4.40 ± 0.82 |
| CCI | 9 | 2.38 ± 0.60 | 3.21 ± 0.30 | 2.68 ± 0.48 | 2.74 ± 0.41 |
| Psi-H | 7 | 2.89 ± 0.57 | 3.72 ± 0.26 | 3.45 ± 0.18 | 3.88 ± 0.18 |
| Psi-L | 7 | 3.21 ± 0.84 | 2.87 ± 0.45 | 2.82 ± 0.45 | 2.51 ± 0.41 |

TABLE 2
Average number of platform crossings in MWM test on PID3 (before
drug treatment) and PIDs 10, 14, 21 (after drug treatment)

| | No. of crossing | | | | |
|---|---|---|---|---|---|
| | n= | Day 3 | Day 10 | Day 14 | Day 21 |
| Naïve | 9 | 9.00 ± 0.83 | 8.33 ± 0.78 | 5.44 ± 0.78 | 6.11 ± 0.77 |
| CCI | 9 | 4.00 ± 0.91 | 4.89 ± 0.59 | 3.89 ± 0.51 | 5.00 ± 0.47 |
| Psi-H | 7 | 4.43 ± 0.69 | 6.00 ± 0.85 | 6.00 ± 0.44 | 6.00 ± 0.66 |
| Psi-L | 7 | 3.57 ± 0.90 | 4.14 ± 0.51 | 4.43 ± 0.61 | 3.29 ± 0.57 |

TABLE 3
Movement speed in MWM test on PID 3 (before drug treatment)
and PIDs 10, 14, 21 (after drug treatment)

| | Velocity (cm/sec) | | | | |
|---|---|---|---|---|---|
| | n= | Day 3 | Day 10 | Day 14 | Day 21 |
| Naïve | 9 | 29.47 ± 0.81 | 28.59 ± 0.55 | 27.19 ± 0.84 | 27.23 ± 1.29 |
| CCI | 9 | 27.00 ± 1.07 | 27.23 ± 0.96 | 27.08 ± 1.10 | 25.93 ± 1.17 |
| Psi-H | 7 | 23.27 ± 1.40 | 25.00 ± 1.57 | 26.38 ± 1.22 | 27.06 ± 1.53 |
| Psi-L | 7 | 26.35 ± 1.44 | 25.79 ± 1.39 | 26.18 ± 0.76 | 26.66 ± 0.76 |

FIG. 4A is a bar graph showing TBI significantly reduced duration stayed in the target zone, FIG. 4B is a bar graph showing the average number of platform crossings, and FIG. 4C is a bar graph showing movement speed on PID3. * denotes two-tailed student's t-test.

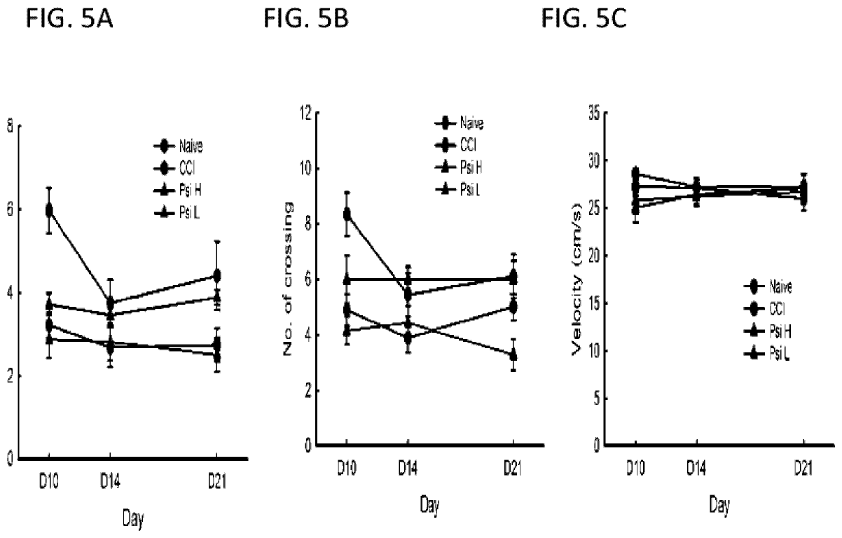

After receiving drug or vehicle treatment from day 4 to day 8, animals were re-evaluated in 60 s probe trials without the escape platform on PID 10, 14, and 21. As seen in FIG. 5, high dose Psilocybin treatment significantly increased the time in the target area and the average number of platform crossings in TBI mice (p values is shown in Table 4-5).

TABLE 4
Significant improvement in Water maze test
after a high dose Psilocybin treatment.

| | Time in the target | No. of |
|---|---|---|
| | area (sec) | crossing |
| | TBI | TBI |
| naive | P < 0.001 | P < 0.001 |
| Psi-H | P = 0.045 | P = 0.011 |
| Psi-L | P = 0.712 | P = 0.237 |

All data were compared to the animals receive TBI and vehicle.
P-value was calculated by a two way ANOVA + posthoc Fisher test.

TABLE 5
Significant improvement in Water maze test after drug treatment.

| | Time in the target | |
|---|---|---|
| | area (sec) | No. of crossing |
| vs | naive | naive |
| T.B.I. | P < 0.001 | P < 0.001 |
| Psi-H | P = 0.011 | P = 0.245 |
| Psi-L | P < 0.001 | P < 0.001 |

All data were compared to the naïve animals.
P-value was calculated by a two way ANOVA + posthoc Fisher test.

The movement velocity was not altered amongst all groups. (see mean and S.E.M. in Table 1-3). CCI or Psilocybin did not significantly alter body weight (Table 6).

TABLE 6
CCI or Psilocybin treatment did not significantly alter body weight

| | n= | body weight (g) |
|---|---|---|
| Naïve | 9 | 35.67 ± 0.53 |
| CCI | 9 | 36.78 ± 0.70 |
| Psi-H | 7 | 34.7 ± 0.87 |
| Psi-L | 7 | 35.71 ± 0.68 |

The bodyweight of animals was measured on PID21

FIGS. 5A, 5B and 5C are graphs showing high dose Psilocybin (PSI) treatment significantly increased the time in the target area and the average number of platform crossings in TBI mice. The velocity of movement was not altered. See also the p-value in Tables 4 and 5.

### 4. Discussion:

In this study, the potential therapeutic effect of Psilocybin in neuronal culture and experimental animals was characterized. It was first demonstrated that Psilocybin did not significantly alter glutamate-mediated loss of MAP2-ir in primary cortical neurons.

Using a TBI mouse model, it was found that post-treatment with Psilocybin improved cognitive function as seen in the Morris water maze test. The cognitive improvement by high dose Psilocybin may be attributed to its reparative action as no significant protection was found in neuronal culture.

### REFERENCES

1. Usona Institute. https://www.usonainstitute.org/psilocybin/
2. WebMD https://www.webmd.com/brain/brain-damage-symptoms-causes-treatments #1
3. Johnson M W and Griffiths R G. Potential therapeutic effects of psilocybin. Neurotherapeutics (2017) 14:734-740.
4. Ly C, Greb A C, Cameron L P, et al. Psychedelics Promote Structural and Functional Neural Plasticity. Cell Rep. 2018 Jun. 12; 23(11):3170-3182. doi: 10.1016/j.celrep.2018.05.022.
5. Scott G and Carhart-Harris R L. Psychedelics as a treatment for disorders of consciousness. Neuroscience of Consciousness. 2019, 5(1): niz003.
6. Takei N and Nawa H. mTOR signaling and its roles in normal and abnormal brain development. Front. Mol. Neurosci., 23 Apr. 2014|https://doi.org/10.3389/fnmol.2014.00028
7. Sampedro F Revenga M, Valle M, Roberto N, Domínguez-Clave E, Elices M, Eduardo Luna L, Crippa J, Hallak J, de Araujo D, Friedlander P, Barker S A, Álvarez E, Soler J, Pascual J C, Feilding A, Riba J. Assessing the psychedelic “after-glow” in ayahuasca users: post-acute neurometabolic and functional connectivity changes are associated with enhanced mindfulness capacities. International Journal of Neuropsychopharmacology (2017) 20(9): 698-711. Apter J. Analeptic of lysergic acid diethylamide (LSD) against phenobarbital. Arch. Ophthal., 59:722-730, 8 1958.
8. Pietrobon D, Striessnig J. Neurobiology of migraine. Nat. Rev. Neuroscience. 2003 4:386-398
9. https://www.who.int/news-room/fact-sheets/detail/headache-disorders
10. https://americanmigrainefoundation.org/resource-library/hemiplegic-migraine/
11. López-Giménez J F, González-Maeso J. Hallucinogens and Serotonin 5-HT2A Receptor-Mediated Signaling Pathways. Curr Top Behav Neurosci. 2018; 36:45-73. doi: 10.1007/7854_2017_478.
12. Hanley N R, Van de Kar L D. Serotonin and the neuroendocrine regulation of the hypothalamic—pituitary-adrenal axis in health and disease. Vitam Horm. 2003; 66:189-255.
13. Marcus D A. Serotonin and its role in headache pathogenesis and treatment. Clin J Pain. 1993 September; 9(3): 159-67.
14. Horvath G A, Selby K, Poskitt K, Hyland K, Waters P J, Coulter-Mackie M, Stockler-Ipsiroglu S G. Hemiplegic migraine, seizures, progressive spastic paraparesis, mood disorder, and coma in siblings with low systemic serotonin.
15. Cephalalgia. 2011 November; 31(15):1580-6. doi: 10.1177/0333102411420584. Epub 2011 Oct. 19.
16. Shenberg E E. Psychedelic-Assisted Psychotherapy: A Paradigm Shift in Psychiatric Research and Development. Front Pharmacol. 2018; 9: 733.
17. Sewell R A, Halpern J H, Pope H G Jr. Response of cluster headache to psilocybin and LSD. Neurology. 2006 Jun. 27; 66(12):1920-2.
18. Qui et al. Abnormal Brain Functional Connectivity of the Hypothalamus in Cluster Headaches. PLOS One. 2013; 8(2): e57896.
19. Leone M, Franzini A, Broggi G, Bussone G. Hypothalamic stimulation for intractable cluster headache: Long-term experience. Neurology 2006:67:150-152.
20. Schulte L H, Allers A, May A. Hypothalamus as a mediator of chronic migraine: evidence from high-resolution IMRI. Neurology. 2017; 88:1-6.

1. A method for the treatment of a brain injury or a migraine in a mammal comprising administering a therapeutically effective amount of psilocybin or a pharmaceutically acceptable salt or solvate thereof, together with one or more pharmaceutically acceptable carriers, diluents and excipients to a mammal in need thereof.

2. The method of claim 1, wherein the mammal is a human.

3. The method of claim 1, wherein the therapeutically effective amount is a dose within the range of about 0.5 mL/day to about 3 mL/day at about 0.5 mM to about 3.0 mM concentration.

4. The method of claim 1, wherein the therapeutically effective amount is a dose of about 0.5 mL/day at about a 2.0 mM concentration.

5. The method of claim 1, wherein the therapeutically effective amount is a dose of about 1.2 mL/day at about a 2.0 mM concentration.

6. The method of claim 1, wherein the therapeutically effective amount is a dose of about 3.0 mL/day at about a 2.0 mM concentration.

7. The method of claim 1, wherein the brain injury is selected from the group consisting of a mild brain injury or traumatic brain injury.

8. The method of claim 7, wherein the brain injury is selected from the group consisting of a concussion, a stroke and Alzheimer's.

9. The method of claim 1, wherein the step of administering includes administering the psilocybin or a pharmaceutically acceptable salt or solvate thereof, together with one or more pharmaceutically acceptable carriers, diluents and excipients, administering to the patient orally.

10. The method of claim 9, wherein the step of administering to the patient orally includes administering buccally or sublingually.

11. The method of claim 9, wherein the step of administering to the patient orally includes administering in as tablets, capsules, powders, granules, lozenges, creams or liquid preparations.

12. (canceled)

13. (canceled)

14. (canceled)

15. (canceled)

16. (canceled)

17. (canceled)

18. (canceled)

19. (canceled)

20. (canceled)
